(19) United States
(12) Patent Application Publication
(10) Pub. No.: US 2007/0232860 A1
(43) Pub. Date: Oct. 4, 2007

(54) ENDOSCOPE

(75) Inventors: Wataru KUBO, Saitama (JP); Masahiro OONO, Saitama (JP); Akira ARIMOTO, Tokyo (JP); Shinichi ARAI, Kanagawa (JP); Koichi SATO, Saitama (JP); Koichi TSUTAMURA, Saitama (JP); Shinichi TAKAYAMA, Tokyo (JP)

Correspondence Address: GREENBLUM & BERNSTEIN, P.L.C., 1950 ROLAND CLARKE PLACE, RESTON, VA, 20191, US

(73) Assignee: PENTAX CORPORATION, Tokyo (JP)

(21) Appl. No.: 11/691,533

(22) Filed: Mar. 27, 2007

(30) Foreign Application Priority Data
Mar. 28, 2006 (JP) 2006-087799

(51) Int. Cl. A61B 1/06

(52) U.S. Cl. 600/160

(57) ABSTRACT

An endoscope comprises an electric scope and a processor. The electric scope has a CMOS sensor and a video-signal emitting unit that outputs an image signal that is imaged by the CMOS sensor and that is converted to a light signal. The processor has a video-signal photo sensor unit that receives light regarding the image signal that is output from the video-signal emitting unit, and performs an image processing operation based on the light regarding the image signal.

### BACKGROUND OF THE INVENTION

1. Field of the Invention

The present invention relates to an endoscope and in particular, a light signal transmitting apparatus.

2. Description of the Related Art

An endoscope that has a light signal transmitting apparatus between the electric scope and the processor, is proposed.

Japanese unexamined patent publication (KOKAI) No. H10-295635 discloses an endoscope that has a light signal transmitting apparatus for transmitting image signals from the endoscope to the processor.

However, because the CCD and the driving circuit for the CCD are arranged at the distal end part of the electric scope, the distal end part becomes large. In the case where the driving circuit for the CCD is arranged in the processor, positive electric power, negative electric power, and a control line for driving the CCD are all necessary, thus requiring a thick cable between the electric scope and the processor.

### SUMMARY OF THE INVENTION

Therefore, an object of the present invention is to provide an apparatus that uses light for transmitting signals without enlarging the distal end part of the electric scope.

According to the present invention, an endoscope comprises an electric scope and a processor. The electric scope has a CMOS sensor and a video-signal emitting unit that outputs an image signal that is imaged by the CMOS sensor and converted to a light signal. The processor has a video-signal photo sensor unit that receives light regarding the image signal output from the video-signal emitting unit, and performs an image processing operation based on the light regarding the image signal.

### BRIEF DESCRIPTION OF THE DRAWINGS

The objects and advantages of the present invention will be better understood from the following description, with reference to the accompanying drawings in which:

FIG. 1 is a block diagram of the endoscope of the first, second, and third embodiments;

FIG. 2 is a side view of the imaging unit in the first embodiment;

FIG. 3 is a top view of the imaging unit in the first embodiment;

FIG. 4 is a top view of the imaging unit in the second embodiment; and

FIG. 5 is a top view of the imaging unit in the third embodiment.

### DESCRIPTION OF THE PREFERRED EMBODIMENTS

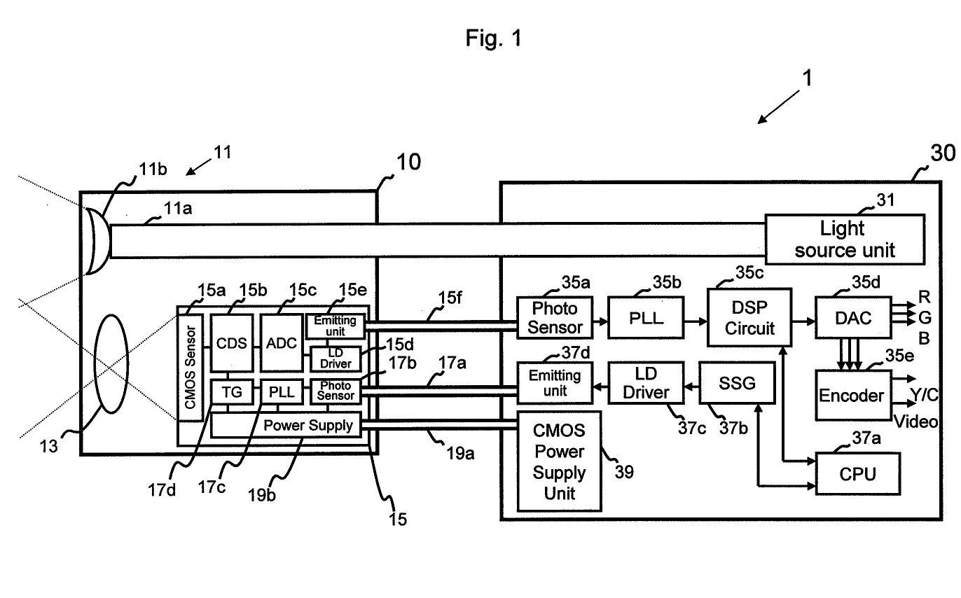

The present invention is described below with reference to the embodiments shown in the drawings. As shown in FIG. 1, an electric endoscope system 1 relating to an embodiment of the present invention is provided with an electric scope 10, and a processor 30.

The electric scope 10 has a lighting unit 11, an objective optical system 13, and an imaging unit 15 at the distal end part of the electric scope 10. The imaging unit 15 images a body (a hollow interior of an organ) etc. that is the photographic subject and is illuminated by the lighting unit 11, through the objective optical system 13.

The lighting unit 11 has a light guide 11a and a lens for lighting 11b.

The imaging unit 15 has a CMOS sensor 15a, a CDS (Correlated Double Sampling) circuit 15b, an ADC (Analog Digital Converter) 15c, a video-signal LD driver 15d, a video-signal emitting unit 15e that is a VCSEL (Vertical Cavity Surface Emitting Laser etc.), a video-signal optical fiber cable 15f, a control-signal optical fiber cable 17a, a control-signal photo sensor unit 17b that is a PD (Photo Diode etc.), a control-signal PLL decoder 17c, a TG (Timing Generator) 17d, a power supply cable 19a, and a power supply unit 19b.

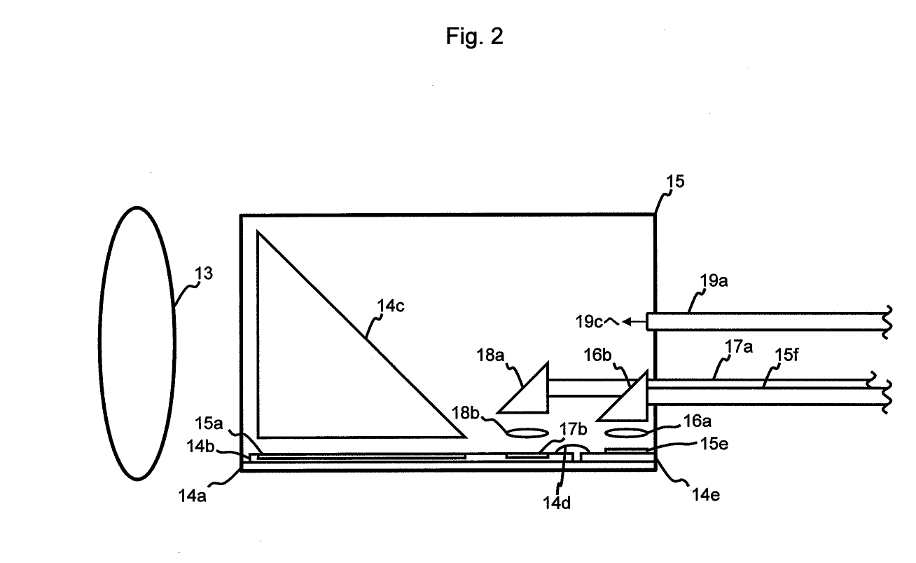

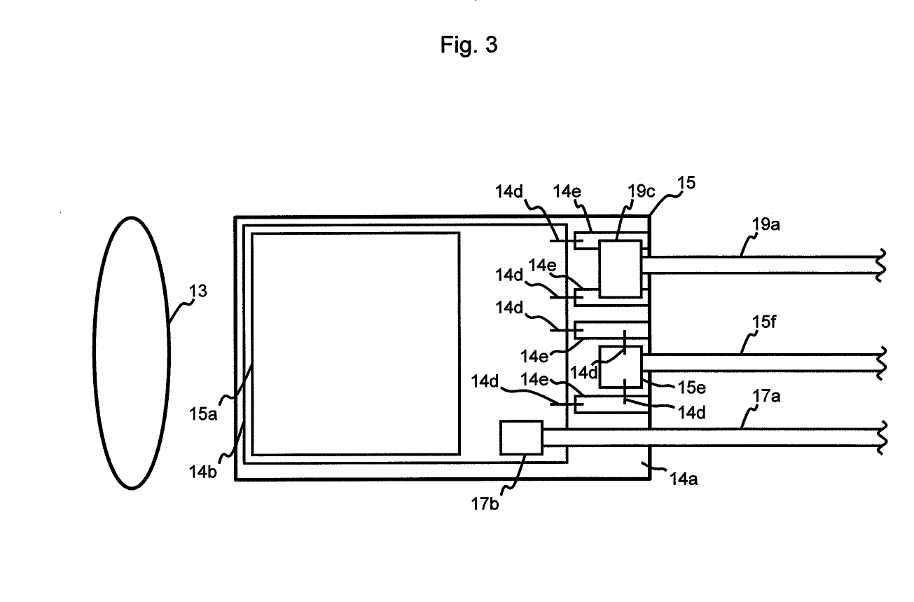

The imaging unit 15 has a ceramic circuit board 14a, a CMOS sensor tip 14b that is a silicon circuit board, an imaging prism 14c, a wire bonding 14d, a lead frame 14e, a video-signal condenser lens 16a, a video-signal prism 16b, a control-signal prism 18a, a control-signal condenser prism 18b, and a bypass condenser 19c as a mounting part (see FIGS. 2 and 3). In FIG. 3, the imaging prism 14c, the video-signal condenser lens 16a, the video-signal prism 16b, the control-signal prism 18a, and the control-signal condenser lens 18b are omitted.

The processor 30 supplies both light and electric power to the electric scope 10, performs an image processing operation on the image signal of the photographing subject imaged by the electric scope 10, and converts the image signal to a video signal that can be displayed on a TV monitor (not depicted).

The processor 30 has a light source unit 31, a video-signal photo sensor unit 35a that is a PD etc., a video-signal PLL decoder 35b, a DSP circuit 35c, a DAC (Digital Analog Converter) 35d, an encoder 35e, a CPU 37a, a SSG (Synchronizing Signal Generator) 37b, a control-signal LD driver 37c, a control-signal emitting unit 37d that is a FP-LD (Fabry-Perot Laser Diode) etc., and a CMOS power supply unit 39.

The light source unit 31 is a lighting circuit that has a xenon lamp light source etc., that illuminates a light that shines upon the photographing subject. The light from the light source unit 31 reaches the photographing subject from the distal end part of the electric scope 10 after traveling through the light guide 11a and the lens for lighting 11b.

The photographing subject is imaged as an optical image through the objective optical system 13 by the CMOS sensor 15a. The optical image is processed by the DSP circuit 35c of the processor 30, after correlated double sampling and A/D conversion have been by the CDS 15b and the ADC 15c, respectively.

In the first embodiment, the CMOS sensor is used as the imaging sensor. Because the amplifier for the CMOS sensor is arranged near the photo sensor (the CMOS sensor) that receives the light, there is a lower occurrence of signal noise compared to when a CCD sensor is used as the imaging sensor.

Further, because a single power supply providing +3.3 volts is used for driving, there is a merit in the small amount of wiring between the distal end part of the electric scope 10 and the processor 30.

Transmission of the image signal from the ADC 15c of the electric scope 10 to the DSP circuit 35c of the processor 30 is accomplished via light. Specifically, the image signal is converted to a digital signal by the ADC 15c, is then converted to an on/off light signal (the light signal) by the video-signal LD driver 15d, whereupon the on/off light signal flashes on and off at the video-signal emitting unit 15e, which in turn is driven by the pulse.

The on/off light signal then travels through the video-signal optical fiber cable 15f before it is received and amplified by the video-signal photo sensor unit 35a. Next, the signal is decoded by the video-signal PLL decoder 35b, after which the decoded signal undergoes an image signal processing operation performed by the DSP circuit 35c.

Therefore, the signal deterioration (loss) between the electric scope 10 and the processor 30 can be reduced in comparison to when an analog electric signal is transmitted from the electric scope 10 to the processor 30.

Further, because a digital electric signal is converted to the light signal that is transmitted from the electric scope 10 to the processor 30, an additional amount of information can be transmitted compared to when an analog electric signal is converted to the transmitted light signal.

For example, in the case that the electric scope 10 has a VGA (640×480≈30 mega pixels) CMOS sensor, a frame rate of 30 frames per second, and a color gradation of 10 bits (1024 steps), the transmitting speed by which the number of pixels, the frame rate, and the color gradation are multiplied is about 92 Mbps. When the analog electric signal is transmitted from the electric scope 10 to the processor 30 by using a thin cable, it is difficult to transmit the image signal at a transmission speed beyond 100 to 200 Mbps without phase delay.

However, when the digital light signal is transmitted in the first embodiment, the image signal can be transmitted without phase delay at high transmission speeds beyond 1 Gbps, corresponding to high density pixels, a high frame rate, and a high color gradation.

After the image processing operation by the DSP circuit 35c and the D/A conversion by the DAC 30d, the video signal that is separated for Y/C by the encoder 35e, the analog RGB component signal, etc., are transmitted to the TV monitor (not depicted). The TV monitor displays them as the image signal.

The CPU 37a controls each part of the electric scope 10 and the processor 30. In particular, trigger signals for AGC (Auto Gain Control), for AE (Auto Exposure), and for obtaining the freeze photograph are transmitted to the electric scope 10 as the command control signal from the CPU 37a through the SSG 37b etc.

Specifically, the SSG 37b generates a pulse signal (a synchronizing signal) controlled by the CPU 37a. The synchronizing signal is converted to the on/off light signal based on the pulse of the control-signal LD driver 37c, and the on/off light signal flashes on and off at of the control-signal emitting unit 37d that is driven by the pulse.

The on/off light signal travels through the control-signal optical fiber cable 17a before it is received and amplified by the control-signal photo sensor unit 17b that has a photo diode. The on/off light signal is then decoded by the control-signal PLL decoder 17c.

The TG 17d outputs a clock pulse based on the signal decoded by the control-signal PLL decoder 17c. The operations of the CMOS sensor 15a, the CDS 15b, and the ADC 15c are performed according to the clock pulse output from the TG 17d.

The CMOS power supply unit 39 of the processor 30 supplies the electric power to the power supply unit 19b of the electric scope 10 through the power supply cable 19a. The power supply unit 19b supplies the electric power to each part of the electric scope 10 such as the imaging unit 15 etc.

In the first embodiment, the supply of the electric power from the processor 30 to the electric scope 10 is delivered through the power supply cable 19a, however the supply of the electric power may be delivered through the light guide 11a. Specifically, a solar battery is arranged at the distal end part of the electric scope 10 that has the CMOS sensor 15a. The light through the light guide 11a is converted to electric energy by the solar battery, and the electric power based on the converted electric energy is supplied to each part of the electric scope 10.

In this construction the CMOS power supply unit 39 and power supply cable 19a are not necessary, thus reducing the diameter required of the cable connecting part of the electric scope 10 with both the processor 30 and the distal end part of the electric scope 10. Furthermore, external noise interference can be mitigated and isolation can be improved between the distal end part of the electric scope 10 and the processor 30, effectively decreasing the potential of an accidental electric shock caused by the high voltage power supply of the xenon lamp of the light source 31.

Next, the mounting part of the CMOS sensor 15a etc., is explained in the first embodiment (see FIGS. 2 and 3).

The CMOS sensor tip 14b is arranged on the ceramic circuit board 14a that is on a plane perpendicular to the lens plane of the objective optical system 13 (parallel to the optical axis of the objective optical system 13).

The CMOS sensor 15a is mounted on the CMOS sensor tip 14b, and images the photographing subject through the objective optical system 13.

The control-signal photo sensor unit 17b is mounted on the CMOS sensor tip 14b, and images (receives) the control signal through the control-signal optical fiber cable 17a.

A part of the dedicated photo diode area of the CMOS sensor tip 14b, may be used for the photo diode of the CMOS sensor 15a, and the other part of the dedicated photo diode area of the CMOS sensor tip 14b, may be used for the photo diode of the control-signal photo sensor unit 17b.

In this case, the manufacturing process can be simplified, and a reduction in cost can be achieved. Specifically, the number of mounting processes for the CMOS sensor 15a and the control-signal photo sensor unit 17b can be reduced compared to when the photo diodes for the CMOS sensor 15a and the control-signal photo sensor unit 17b are mounted with the separate (photo printing) processes.

For example, two photo diodes can be mounted by using one photo printing process and by masking, effectively reducing costs compared to when the two photo diodes are mounted with an alternative manufacturing (photo printing) process.

Further, because the photo diodes of the CMOS sensor 15a and the control-signal photo sensor unit 17b are mounted at the same time, the number of position-adjusting processes can be reduced.

The CDS 15b, the ADC 15c, the control-signal PLL decoder 17c, and the TG 17d are mounted on the CMOS sensor tip 14b (they are not depicted in FIGS. 2 and 3). Therefore, the CMOS sensor 15a, the CDS 15b, the ADC 15c, the control-signal photo sensor unit 17b, the control-signal PLL decoder 17c, and the TG 17d are each mounted with the same manufacturing process.

The incident optical path through the objective optical system 13 is deflected toward the CMOS sensor 15a by the imaging prism 14c.

The transmitted optical path through the control-signal optical fiber cable 17a is deflected toward the control-signal photo sensor unit 17b by the control-signal prism 18a, and condensed by the control-signal condenser lens 18b.

The video-signal emitting unit 15e is connected with wire bonding 14d to the lead frame 14e that is attached to the ceramic circuit board 14a. The light emitted by the video-signal emitting unit 15e is condensed by the video-signal condenser lens 16a, and the condensed optical path is deflected by the video-signal prism 16b toward the imaging unit 15 end (the incident plane) of the video-signal optical fiber cable 15f by the video-signal prism 16b.

The CMOS sensor tip 14b is connected to the lead frame 14e with wire bonding 14d.

The power supply cable 19a is connected to the bypass condenser 19c on the lead frame 14e that is attached to the ceramic circuit board 14a.

The CMOS sensor 15a etc. can be arranged in one plane on a circuit board (the ceramic circuit board 14a) by deflecting the transmitted optical path perpendicularly, with the imaging prism 14c, the video-signal prism 16b, and the control-signal prism 18a.

The distal end part of the electric scope 10 is approximately 10 mm in diameter. In consideration of the arrangement of the nozzle, the light guide 11a, and the throat of the forceps, it is desirable for the part housing the imaging unit 15, upon which the CMOS sensor 15a etc. are mounted, to have an appropriate shape and size so that it does not extend beyond the objective optical system 13 that is roughly 4 mm in diameter.

It is necessary to mount the peripheral circuits, such as the CDS 15b etc., near the CMOS sensor when it functions as the imaging sensor. In the first embodiment, however the ceramic circuit board 14a holding these peripheral circuits is oriented perpendicular to the lens plane of the objective optical system 13. Therefore, the peripheral circuits must be arranged accordingly so that the part housing the imaging unit 15 does not extend beyond the lens diameter of the objective optical system 13.

Next, the second embodiment is explained. The mounting in the second embodiment is different from that in the first embodiment; the points that differ from the first embodiment are explained as follows.

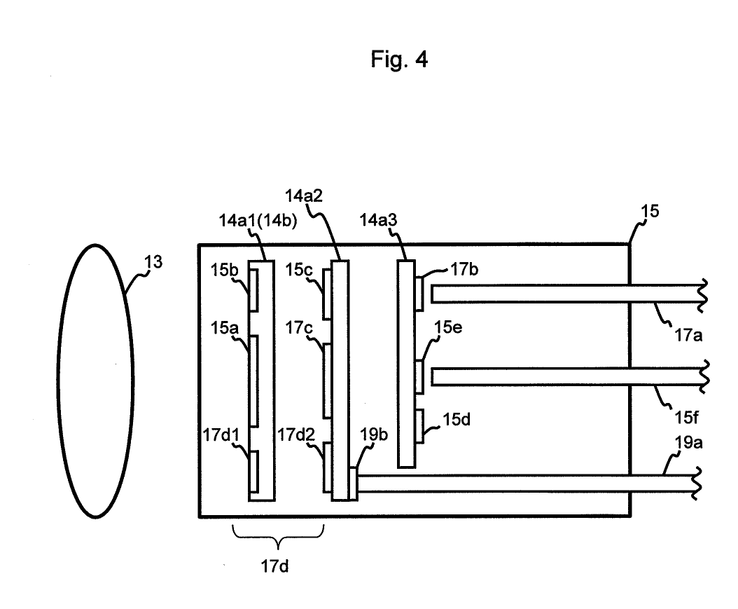

The imaging unit 15 in the second embodiment has a first circuit board 14a1, a second circuit board 14a2, a third circuit board 14a3, and a CMOS sensor tip 14b (see FIG. 4). The TG 17d in the second embodiment has a sub TG 17d1 and a main TG 17d2.

The first, second, and third circuit boards 14a1, 14a2, and 14a3 are laminated circuit boards that are parallel to the lens plane of the objective optical system 13 and arranged in order from the objective optical system 13 side of the imaging unit 15.

The CMOS sensor tip 14b is mounted on the first circuit board 14a1 and on the same side as the objective optical system 13. The CMOS sensor 15a is mounted on the CMOS sensor tip 14b, and images the image-formed photographing subject through the objective optical system 13.

The CDS 15b and the sub TG 17d1 are mounted on the CMOS sensor tip 14b. Therefore, the CMOS sensor 15a, the CDS 15b, and the sub TG 17d1 are each mounted with the same manufacturing process.

The sub TG 17d1 converts a clock pulse that is output from the main TG 17d2 to a clock pulse for the CMOS sensor 15a and the CDS 15b, and then outputs the converted clock pulse to the CMOS sensor 15a and the CDS 15b.

In the second embodiment, because the CMOS sensor 15a needs an accurate control of read out, the sub TG 17d1 that controls the timing of reading out is arranged near the CMOS sensor 15a, making it easier to adjust the timing control of the start and end points of reading out.

Further, because both the interconnect and routing lengths to avoid phase delay can be restrained, the enlargement of the circuit board can be prevented.

Further, if the number of pixels of the CMOS sensor 15a is increased in the future, the phase delay can be restrained and the speed of reading can be held constant, even if the operational speed is increased.

The ADC 15c, the control-signal PLL decoder 17c, the main TG 17d2, and the power supply unit 19b are mounted on the second circuit board 14a2. The main TG 17d2 outputs a clock pulse (a timing pulse) for the ADC 15c etc.

The video-signal emitting unit 15e, the video-signal LD driver 15d, and the control-signal photo sensor unit 17b are mounted on the third circuit board 14a3 and on the side opposite from the objective optical system 13.

An end (the incident plane) of the video-signal optical fiber cable 15f faces an emitting plane of the video-signal emitting unit 15e.

An end (an exit plane) of the control-signal optical fiber cable 17a faces the photo sensing plane of the control-signal photo sensor unit 17b.

It is necessary to mount the peripheral circuits, such as the CDS 15b etc., near the CMOS sensor when it functions as the imaging sensor. In the second embodiment, however, the laminated circuit boards holding these peripheral circuits are oriented perpendicular to the optical axis of the objective optical system 13. Therefore, the peripheral circuits must be arranged accordingly so that the part housing the imaging unit 15 does not extend beyond the lens diameter of the objective optical system 13.

Next, the third embodiment is explained. The optical fiber cable in the third embodiment is different from that in the second embodiment, the points that differ from the second embodiment are explained as follows.

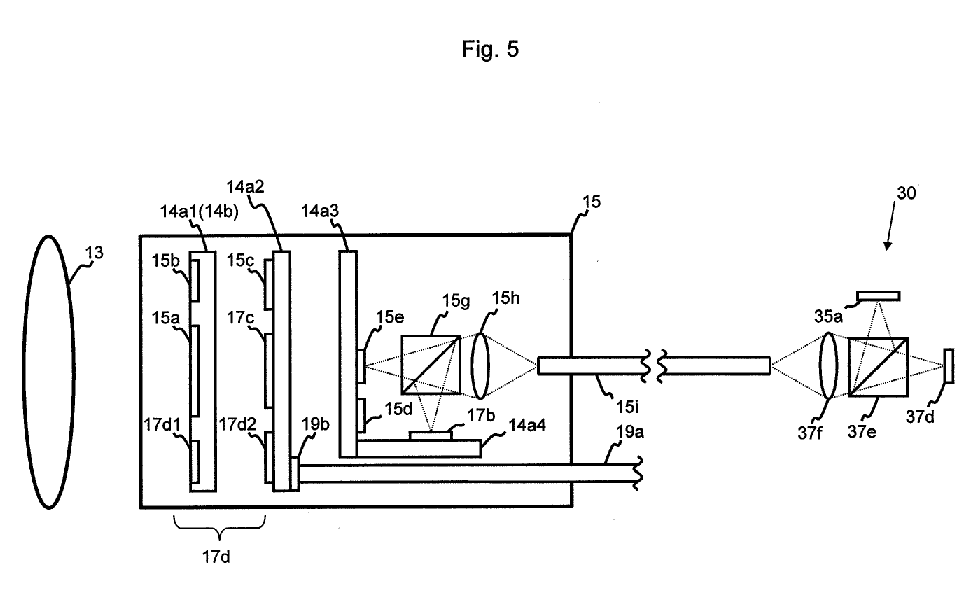

The imaging unit 15 in the third embodiment has a first circuit board 14a1, a second circuit board 14a2, a third circuit board 14a3, a fourth circuit board 14a4, a CMOS sensor tip 14b, a first polarizing mirror 15g, a first condenser lens 15h, and an optical fiber cable 15i substituting for the video-signal optical fiber cable 14f and the control-signal fiber cable 17a (see FIG. 5).

The TG 17d in the third embodiment has a sub TG 17d1 and a main TG 17d2. The processor 30 in the third embodiment further has a second polarizing mirror 37e and a second condenser lens 37f.

The optical fiber cable 15i is used to transmit video-signals from the electric scope 10 to the processor 30, and to transmit control-signals from the processor 30 to the electric scope 10.

The first, second, and third circuit boards 14a1, 14a2, and 14a3 are laminated circuit boards that are parallel to the lens plane of the objective optical system 13 and arranged in order from the objective optical system 13 side of the imaging unit 15. The fourth circuit board 14a4 is arranged in perpendicular to the third circuit board 14a3.

The construction of the first and second circuit boards 14a1 and 14a2 in the third embodiment is the same as that in the second embodiment.

The video-signal emitting unit 15e and the video-signal LD driver 15d are mounted on the third circuit board 14a3 on the side opposite from the objective optical system 13.

The electric scope 10 end of the optical fiber cable 15i faces the first condenser lens 15h, and faces the emitting plane of the video-signal emitting unit 15e through the first polarizing mirror 15g.

The first polarizing mirror 15g has a polarizing mirror (WDM: Wavelength Division Multiplexing) that transmits the light of a video signal output from the video-signal emitting unit 15e, and reflects the light of a control signal output from the optical fiber cable 15i. The wavelength of light from the video signal is set different from the wavelength of light from the control signal. For example, the light of the video signal, which has more information quantity than that of the control signal, is set in the infrared spectrum that has approximately 850 nm wavelengths, whereas the light of the control signal is set in the red spectrum that has approximately 680 nm wavelengths.

The first condenser lens 15h condenses the light of the video signal that is transmitted from the video-signal emitting unit 15e to the electric scope 10 end of the optical fiber cable 15i, and condenses the light of the control signal that is transmitted from the electric scope 10 end of the optical fiber cable 15i to the control-signal photo sensor unit 17b through the first polarizing mirror 15g.

The control-signal photo sensor unit 17b is mounted on the fourth circuit board 14a4, inside of the imaging unit 15, and is arranged where the control-signal photo sensor unit 17b can receive the light of a control signal that is reflected by the first polarizing mirror 15g.

The video-signal photo sensor unit 35a is arranged where the video-signal photo sensor unit 35a can receive the light of a video signal that is reflected by the second polarizing mirror 37e.

The control-signal emitting unit 37d is arranged where the control-signal emitting unit 37d faces the processor 30 end of the optical fiber cable 15i through the second polarizing mirror 37e and the second condenser lens 37f.

The second polarizing mirror 37e has a polarizing mirror (WDM: Wavelength Division Multiplexing) that transmits the light of a control signal that is output from the control-signal emitting unit 37d, and that reflects the light of a video signal that is output from the optical fiber cable 15i.

The second condenser lens 37f condenses the light of a control signal that is output from the control-signal emitting unit 37d and transmitted through the second polarizing mirror 37e to the processor 30 end of the optical fiber cable 15i, and condenses the light of a video signal that is transmitted from the processor 30 end of the optical fiber cable 15i to the video-signal photo sensor unit 35a through the second polarizing mirror 37e.

In the third embodiment, the optical fiber cable is shared for transmitting both the video signal from the electric scope 10 and the control signal from the processor 30 so that the diameter of the cable of the electric scope 10 can be minimized, thus enabling greater flexibility in the cable while reducing the load on the patient.

Although the embodiments of the present invention have been described herein with reference to the accompanying drawings, obviously many modifications and changes may be made by those skilled in this art without departing from the scope of the invention.

The present disclosure relates to subject matter contained in Japanese Patent Application No. 2006-087799 (filed on Mar. 28, 2006) which is expressly incorporated herein by reference, in its entirety.

1. An endoscope comprising:
an electric scope that has a CMOS sensor and a video-signal emitting unit that outputs an image signal that is imaged by said CMOS sensor and that is converted to a light signal; and
a processor that has a video-signal photo sensor unit that receives light regarding said image signal that is output from said video-signal emitting unit, and that performs an image processing operation based on said light regarding said image signal.

2. The endoscope according to claim 1, wherein said electric scope has an ADC that converts said image signal that is imaged by said CMOS sensor to a digital signal; and
said video-signal emitting unit outputs said image signal that is converted to said digital signal by said ADC and that is converted to said light signal.

3. The endoscope according to claim 2, wherein said processor has a control-signal emitting unit that outputs a control signal that is converted to a light signal; and
said electric scope has a control-signal photo sensor unit that receives light regarding said control signal that is output from said control-signal emitting unit, and has a timing generator that outputs a clock pulse to said CMOS sensor and said ADC based on said light regarding said control signal.

4. The endoscope according to claim 3, wherein said electric scope has a video-signal cable and a control-signal cable that are separate from each other;
said video-signal cable transmits said light regarding said image signal that is output from said video-signal emitting unit; and
said control-signal cable transmits said light regarding said control signal that is output from said control-signal emitting unit.

5. The endoscope according to claim 3, wherein said electric scope has an imaging prism that deflects an incident optical path toward said CMOS sensor, and has a control-signal prism that deflects an incident optical path toward said control-signal photo sensor unit; and
said CMOS sensor, said ADC, said timing generator, and said control-signal photo sensor unit are mounted on the same CMOS sensor tip.

6. The endoscope according to claim 5, wherein a part of the photo diode area of said CMOS sensor tip, is used for a photo diode of said CMOS sensor, and the other part of the photo diode area of said CMOS sensor tip, is used for a photo diode of said control-signal photo sensor unit.

7. The endoscope according to claim 5, wherein said electric scope has a video-signal prism that deflects an output optical path from said video-signal emitting unit regarding said image signal; and
said CMOS sensor tip and said video-signal emitting unit are arranged on a circuit board that consists of one plane.

8. The endoscope according to claim 3, wherein said timing generator has a sub TG that outputs said clock pulse to said CMOS sensor, and has a main TG that outputs a timing pulse to said ADC;
said electric scope has a first circuit board, a second circuit board, and a third circuit board;
said CMOS sensor and said sub TG are arranged on said first circuit board;
said ADC and said main TG are arranged on said second circuit board;
said video-signal emitting unit and said control-signal photo sensor unit are arranged on said third circuit board; and
said first, second, and third circuit boards are arranged in order from the distal end part of said electric scope.

9. The endoscope according to claim 3, wherein said electric scope has a cable that transmits said light regarding said image signal that is output from said video-signal emitting unit and said light regarding said control signal that is output from said control-signal emitting unit, and has a first polarizing mirror that transmits through said light regarding said image signal and that reflects said light regarding said control signal; and
said processor has a second polarizing mirror that reflects said light regarding said image signal and that transmits through said light regarding control signal.

10. The endoscope according claim 1, wherein said electric scope supplies electric power to said CMOS sensor based on light from outside of said electric scope.

11. The endoscope according to claim 1, wherein said electric scope supplies electric power to each part of the distal end part of said electric scope that has said CMOS sensor, based on light from outside of said electric scope.
